(19) United States
(12) Patent Application Publication
(10) Pub. No.: US 2022/0082368 A1
(43) Pub. Date: Mar. 17, 2022

(54) PROBE WITH OPTIMIZED FOCAL DEPTH, WORKING DISTANCE AND AXIAL LIGHT INTENSITY UNIFORMITY

(71) Applicant: ZHEJIANG UNIVERSITY, Hangzhou (CN)

(72) Inventors: Zhihua DING, HANGZHOU (CN); Jianrong QIU, HANGZHOU (CN); Zhiyi LIU, HANGZHOU (CN)

(21) Appl. No.: 17/535,654

(22) Filed: Nov. 25, 2021

Related U.S. Application Data
(63) Continuation of application No. PCT/CN2020/088523, filed on Apr. 30, 2020.

(30) Foreign Application Priority Data
Sep. 26, 2019 (CN) 201910917336.7

(51) Int. Cl. G01B 9/02055; A61B 5/00; G01B 9/02015

(52) U.S. Cl. CPC G01B 9/02063; A61B 5/0084; G01B 9/02023; A61B 5/0066

(57) ABSTRACT

A probe with optimized focal depth, working distance and axial light intensity uniformity, including a single-mode fiber for guiding light, a first gradient index fiber for improving light propagation efficiency and regulating mode energy, a large core fiber for generating mode interference field (MIF) and regulating an mode phase difference, a second gradient index fiber and a no-core fiber for magnifying the MIF, and a third gradient index fiber for focusing.

### CROSS-REFERENCE TO RELATED APPLICATIONS

This application is a continuation of International Patent Application No. PCT/CN2020/088523 with a filing date of Apr. 30, 2020, designating the United States, now pending, and further claims priority to Chinese Patent Application No. 201910917336.7 with a filing date of Sep. 26, 2019. The content of the aforementioned applications, including any intervening amendments thereto, are incorporated herein by reference.

### TECHNICAL FIELD

This disclosure relates to the field of OCT (Optical Coherence Tomography), and in particular relates to a probe that utilizes modal interference in optical fibers to simultaneously achieve focal depth extension, WD (working distance) extension, and optimization of axial light intensity uniformity.

### BACKGROUND

OCT is an important imaging method due to the capability of obtaining high-resolution three-dimensional structural and/or functional information of internal organs of a living body through an endoscopic probe (Herz, P., et al., Ultrahigh resolution optical biopsy with endoscopic optical coherence tomography. Opt Express, 2004. 12(15): p. 3532-42). Compared with conventional optical imaging methods, the axial resolution of OCT has no concern with its lateral resolution, but depends on the spectral bandwidth of the light source. With the most advanced broadband light source, an axial resolution can reach 1-5 microns (Drexler, W., et al., Ultrahigh-resolution ophthalmic optical coherence tomography. Nat Med, 2001. 7(4): p. 502-7). However, if the lateral resolution is increased to the same magnitude, the effective imaging range of OCT will be limited by the extremely short focal depth of the beam due to rapid divergence of the beam near focal point.

In order to solve the contradiction between the lateral resolution and the focal depth, a variety of solutions have been proposed and an order of magnitude extension of the focal depth is achieved, such as digital focus (Ralston, T. S., et al., Interferometric synthetic aperture microscopy. Nat Phys, 2007. 3(2): p. 129-134. AND Bo, E., et al., Depth-of-focus extension in optical coherence tomography via multiple aperture synthesis. Optica, 2017. 4(7): p. 701-706), dynamic focus (Qi, B., et al., Dynamic focus control in high-speed optical coherence tomography based on a microelectromechanical mirror. Optics Communications, 2004. 232(1-6): p. 123-128) and quasi-optical needle focus (Bao, W., et al., Quasi-needle-like focus synthesized by optical coherence tomography. Opt Lett, 2017. 42(7): p. 1385-1388). However, some of the above methods require phase stability, some require mechanical scanning, and some require two optical paths to realize illumination and detection separately. Therefore, these methods are difficult to apply to miniaturized probes. Miniature axicon made by chemical etching (Tan, K M., et al., In-fiber common-path optical coherence tomography using a conical-tip fiber. Optics Express, 2009. 17(4): p. 2375-2384) or grinding and polishing (Wang, W., et al., Miniature all-fiber axicon probe with extended Bessel focus for optical coherence tomography. Opt Express, 2019. 27(2): p. 358-366) and miniature binary phase plates (Kim, J., et al., Endoscopic micro-optical coherence tomography with extended depth of focus using a binary phase spatial filter. Optics Letters, 2017. 42(3): p. 379-382) made by soft lithography have been used to extend the focal depth of the probe. However, compared with the desktop system, these miniature optical elements have a very limited extension of the focal depth of the probe. On the other hand, a solution based on phase mask that does not require other processing techniques except cutting and fusion splicing a series of optical fibers has been reported (Lorenser, D., X. Yang, and D. D. Sampson, Ultrathin fiber probes with extended depth of focus for optical coherence tomography. Opt Lett, 2012. 37(10): p. 1616-8). But the solution based on phase mask requires a very high cutting accuracy of the optical fiber. Another method that achieves focal depth extension in an all-fiber probe uses the higher-order modes (Zhu, X., et al., Generation of controllable nondiffracting beams using multimode optical fibers. Applied Physics Letters, 2009. 94(20): p. 201102. AND Yin, B., et al., Extended depth of focus for coherence-based cellular imaging. Optica, 2017. 4(8): p. 959-965) in step-index fibers, but the deconstructing interference in the focal depth region causes an uneven distribution of the light intensity of the output beam in the axial direction, which has an adverse effect on the overall imaging quality. Recently, the focal depth of the all-fiber probe was extended by modulating the mode interference field on the entrance pupil of the fiber lens (Qiu, J R., et al., All-fiber probe for optical coherence tomography with an extended depth of focus by a high-efficient fiber-based filter. Optics Communications, 2018. 413: p. 276-282), but its working distance is limited.

### SUMMARY

Aiming at the deficiencies of the prior art, the present disclosure provides a probe with optimized focal depth, working distance and axial light intensity uniformity.

A probe with optimized focal depth, working distance and axial light intensity uniformity, including a single-mode fiber, a GIF1 (first gradient index fiber), a LCF (large core fiber) configured to adjust an mode phase difference, a GIF2 (second gradient index fiber), a no-core fiber and a GIF3 (third gradient index fiber);

the single-mode fiber, the first gradient index fiber, the large core fiber, the second gradient index fiber, the no-core fiber, and the third gradient index fiber are fused and spliced in sequence; the second gradient index fiber magnifies a MIF (Mode Interference Field) on the end of the large core fiber to the entrance pupil of the third gradient index fiber, a length of the no-core fiber satisfies a requirement that the magnified MIF after imaging fills the entrance pupil of the third gradient index fiber. The magnified MIF serves as the optical pupil filter of the probe, which is conducive to the extension of the focal depth and the extension of the working distance.

Preferably, the MIF as a result of mode interference in the large core fiber is generated at the end of the large core fiber, the MIF is tuned by adjusting the length of the first gradient index fiber and the length of the large core fiber, the first gradient index fiber manipulates the mode power of the MIF, and the large core fiber adjusts the mode phase difference of the MIF.

Preferably, an outer diameter of each fiber is the same as that of a standard single-mode optical fiber, which ensures a stable and reliable structure of the spliced probe. The monolith probe is equivalent to a single-mode optical fiber, which is convenient for various endoscopic scenarios.

Preferably, a length of the third gradient index fiber used for focusing realizes the required lateral resolution dependent on the application.

The mode phase difference can be adjusted by the length of the LCF, which is the key to optimize the uniformity of light intensity on the axial direction of the output beam.

The GIF1 is used to improve light propagation efficiency and adjust mode power. For example: when the length of the GIF1 is a quarter of its pitch, the coupling efficiency between the GIF1 and the following LCF is relatively high, and mainly the LP01 mode is excited with negligible LP02 mode, so that the mode interference in LCF is negligible; when the length of GIF1 is 0 (there is no GIF1), the light energy tends to be distributed in the higher-order mode of the LCF, but the direct coupling of the SMF and LCF may cause higher insertion loss.

In the present disclosure, a design that simultaneously extends the focal depth, extends the working distance, and optimizes the uniformity of the axial light intensity of the output beam by a fiber-based pupil filter is proposed. The MIF magnified by the lens instead of the MIF directly expanded by diffraction is used as the final optical pupil filter. The uniformity of the axial light intensity of the output beam is optimized by adjusting the mode phase difference. The probe includes the single-mode fiber for guiding light, the first gradient index fiber for improving light propagation efficiency and regulating mode energy, the large core fiber for generating MIF and regulating the mode phase difference, the second gradient index fiber and the no-core fiber for magnifying MIF, and the third gradient index fiber for focusing.

Compared with the prior arts, the present disclosure achieves the following beneficial effects:

1. Compared with digital focus, dynamic focus and quasi-optical needle focus, the present disclosure does not require phase stability, does not need mechanical scanning, and uses the same optical path for illumination and detection, which is beneficial to the miniaturization of the probe.

2. Compared with the methods based on miniature axicon and miniature binary phase plate, the production of the probe of the present application is compatible with existing all-in-fiber probe fabrication techniques, and no other processing technology is required except cutting and fusion splicing a series of optical fibers. In addition, the optical fibers adopted by the present disclosure have the same cladding diameter (only 125 μm) as standard single-mode optical fibers, so that the probe formed by fusion splicing has a reliable structure.

3. Compared with the method based on the phase mask, the present disclosure allows larger manufacturing errors, which reduces the manufacturing cost.

4. Compared with the method based on higher-order modes, the present disclosure further optimizes the uniformity of the axial light intensity of the output beam by adjusting the mode phase difference.

5. Compared with existing method based on fiber optical pupil filter, the magnified mode interference field instead of the diffracted one is adopted as the final pupil filter in the present disclosure. The present disclosure has both enhanced depth of focus and working distance, and the axial uniformity of the output beam is further optimized by tuning the mode phase difference.

### BRIEF DESCRIPTION OF THE DRAWINGS

FIG. 1(a) is a structural schematic diagram of a conventional probe; SMF, single-mode fiber, NCF, no-core fiber, GIF, gradient index fiber;

FIG. 1(b) is the probe structure of the present disclosure; SMF, single-mode fiber, GIF1, first gradient index fiber; LCF, large core fiber, GIF2, second gradient index fiber; NCF, no-core fiber; GIF3, third gradient index fiber; P1 and P2 are a pair of conjugate focal planes of GIF2;

FIG. 2 shows the simulated field intensity of the output beams in air normalized to the peak intensity in SMF for six typical cases of the designed probe.

FIG. 3(a) is the microscope image of the fabricated conventional probe; SMF, single-mode fiber, NCF, no-core fiber, GIF, gradient index fiber;

FIG. 3(b) is the microscope image of the fabricated of the present disclosure; SMF, single-mode fiber, GIF1, first gradient index fiber, LCF, large core fiber, GIF2, second gradient index fiber; NCF, no-core fiber, GIF3, third gradient index fiber;

FIG. 4 is the schematic of the probe-based swept source OCT system;

FIG. 5 (a) shows the full width half maximum (FWHM) diameter of the output beam from the conventional probe versus depth in air, WD represents working distance;

FIG. 5 (b) shows the full width half maximum (FWHM) diameter of the output beam from the probe of the present disclosure versus depth in air; WD represents working distance;

FIG. 5(c) is the cross-sectional reflectivity profiles of the resolution target at the probe's foci imaged by the conventional probe;

FIG. 5(d) is the cross-sectional reflectivity profiles of the resolution target at the probe's foci imaged by the present disclosure.

### DETAILED DESCRIPTION OF PREFERRED EMBODIMENTS

The present disclosure will be described in detail below with reference to the drawings and embodiments.

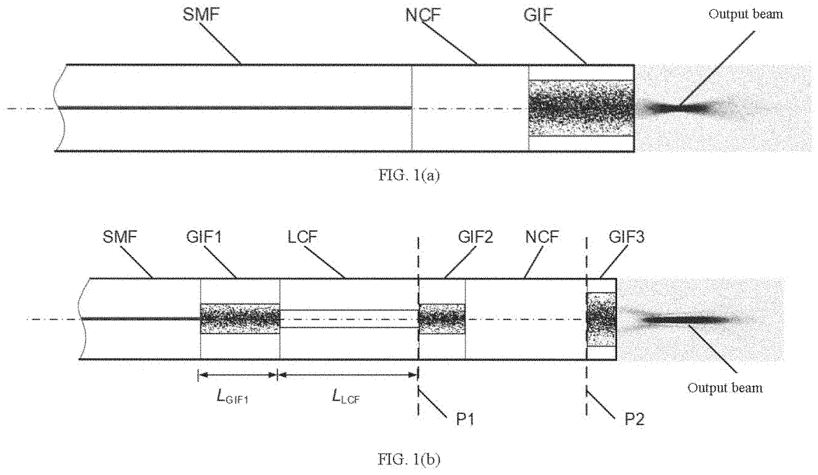

As shown in FIG. 1(a), a conventional all-fiber probe includes a single-mode fiber (SMF), a no-core fiber (NCF), and a gradient index fiber (GIF). The SMF is used for light delivery, the NCF is used for beam expansion, and the GIF is used for beam focusing. In order to extend a focal depth, a series of fiber sections GIF1-LCF-GIF2 are inserted between the SMF and NCF, as shown in FIG. 1(b). The first gradient index fiber (GIF1) is used to manipulate the modes excited in the following large core fiber (LCF). Without GIF1, the incident power will be distributed among high order modes with increased insertion loss due to mismatched numerical aperture between the LCF and the SMF. Light field in the LCF can be decomposed into linearly polarized (LP) modes with different propagation constants. Due to the symmetry of the probe structure and the limited V-number of the LCF, only LP₀₁ and LP₀₂ modes with cut-off frequency lower than the V-number are actually excited and can steadily propagate in the LCF. The interference of LP₀₁ mode and LP₀₂ mode forms a mode interference field (MIF) at the end of LCF. This MI field (MIF) functions as a pupil filter for DOF extension, and is regulable by length tuning of the LCF. On the other hand, to increase the WD of the probe, the beam size of this MIF needs to be expanded. One way of MIF expansion can be realized by direct propagation of this MIF in the NCF. However, due to light diffraction in the homogeneous medium, the evolved MIF is not exactly an expanded version of the original MIF, and its performance as a pupil filter might be inferior to the expectation. The second way of MIF expansion can be done by magnified imaging. Hence, the GIF2 is utilized to relay the MIF at the LCF-GIF2 interface (labeled as P1 in FIG. 1(b)) onto the entrance pupil (labeled as P2 in FIG. 1(b)) of the GIF3 with magnification.

TABLE 1
Parameters of optical fibers used by
the probe (wavelength is 1.3 μm)

| | | GIF1/ | | | GIF/ |
|---|---|---|---|---|---|
| | SMF | GIF2 | LCF | NCF | GIF3 |
| core diameter/μm | | 50 | 25 | — | 62.5 |
| cladding diameter/μm | 125 | 125 | 125 | 125 | 125 |
| mode field diameter/μm | 9.2 | — | | | |
| refractive index | | 1.4607 | 1.4469 | 1.4469 | 1.4728 |
| in the fiber axis | | | | | |
| cladding refractive index | | 1.4469 | 1.4434 | 1.4469 | 1.4469 |

It is speculated that the MIF at the end of the LCF and the way of MIF expansion might affect the output beam of the probe. In view of the excited modes allowable in the LCF, three situations are considered, including mainly LP₀₁ mode with negligible mode interference and two mixed LP₀₁ and LP₀₂ modes with significant mode interference but distinct mode phase difference at the end of the LCF. These three situations of allowable modes in combination with two choices of MIF expansion result in six typical cases for the designed probe. Table 1 lists the parameters of each fibers in the probe (wavelength is 1.3 μm). Table 2 lists the fiber lengths and the characteristics of the output beam under six typical cases, where magnified MIF with the GIF2 is adopted in cases I (negligible mode interference), II and III (significant mode interference but distinct mode phase difference), while diffracted MIF without the GIF2 is applied in cases IV (negligible mode interference), V and VI (significant mode interference but distinct mode phase difference) for comparison.

TABLE 2
Fiber lengths and characteristics of the
output beam under six typical cases of
the designed probe

| | I | II | III | IV | V | VI |
|---|---|---|---|---|---|---|
| L_{Gif1} (μm) | 285 | 0 | 485 | 285 | 0 | 485 |
| L_{LCF2} (μm) | 820 | 890 | 1250 | 1315 | 1310 | 1115 |
| L_{GIF2} (μm) | 390 | 390 | 390 | 0 | 0 | 0 |
| L_{NCF} (μm) | 300 | 300 | 300 | 300 | 300 | 300 |
| L_{GIF3} (μm) | 160 | 160 | 160 | 160 | 160 | 160 |
| MBD (μm) | 5.2 | 5.8 | 4.2 | 5.5 | 5.0 | 4.5 |
| DOF (μm) | 190 | 305 | 240 | 175 | 290 | 195 |
| WD (μm) | 185 | 187 | 200 | 122 | 150 | 142 |
| NDOFG | 1.16 | 1.50 | 2.25 | 0.96 | 1.92 | 1.59 |

The normalized depth of focus gain (NDOFG) of the output beam from the probe is expressed as:

$$NDOFG = \frac{0.1274 λ \cdot DOF}{n \cdot {FHWM}^{2}} ,$$

wherein λ is a central wavelength, n is a refractive index of a medium outside the probe, the beam diameter (FHWM) is defined as the full width at half maximum of the lateral light intensity of the beam, and a depth of focus (DOF) is defined as a depth range where the beam diameter is less than twice its minimum value. For Gaussian beams, NDOFG equal to 1.

The mode phase difference at the end of the LCF is:

$$Δϕ = {Δϕ}_{0} ( {L}_{GIF 1} ) + Δ \overline{β} {L}_{LCF} ,$$

wherein Δϕ₀ is an initial mode phase difference at the GIF1-LCF interface. Δβ̅ is a difference between the propagation constant of the LP₀₂ mode and propagation constant of LP₀₁ mode in the LCF, L_{GIF1} and L_{LCF} is a length of the GIF1 and the LCF respectively.

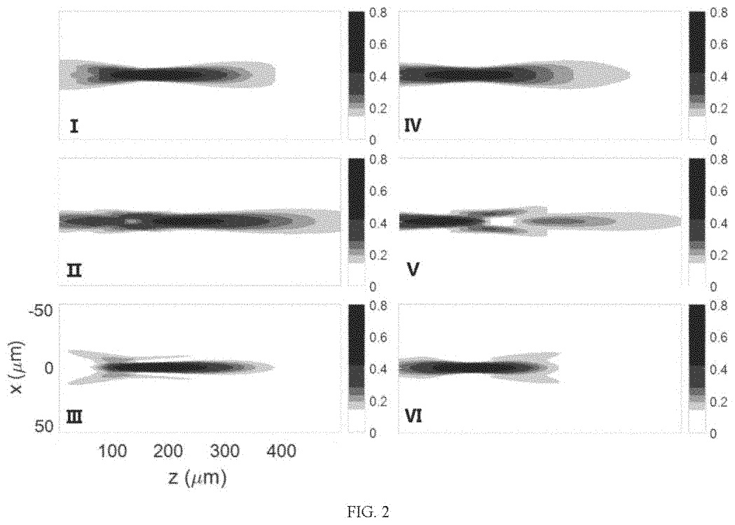

The lengths for GIF1 and LCF in six cases listed in Table 2 are chosen with high coupling efficiency to realize negligible two-mode interference, or significant two-mode interference with distinct mode phase difference. For cases I and IV, a quarter-pitch-length of the GIF1 is chosen to excite mainly the LP₀₁ mode with negligible LP₀₂ mode, while the length of the LCF with insignificant effect on the MIF is determined to realize Δϕ with π difference than that in cases II and V. The lengths of the GIF1 for cases II, III, V and VI are chosen to obtain significant two-mode interference aiming to the maximized DOF gain. However, Δϕ at the end of the LCF for cases II and V are distinct from cases III and VI, which might induce different axial intensity profile of the output beam. The lengths of the NCF are chosen so that the diffracted MIF can fully fill the aperture of the following GIF3. The lengths of the GIF2 are determined by the conjugated relation between P1 and P2. The lengths of GIF3 are chosen to yield an output beam with ˜5 μm minimal beam diameter (MBD). To demonstrate controllable manipulation of the output beam, light field simulations on six typical cases of the designed probe are conducted. Beam propagation method and angular spectrum method are adopted for segmented fibers and homogeneous medium (NCF, air), respectively. The field intensity distributions of output beams in air for six cases are shown in FIG. 2, with characteristics listed in Table 2. Significant DOF gain (≥1.5) is realized in cases II, III, V and VI where mode interference is significant. Also, destructive interference in the focus region is observed in cases II and V but not available in cases III and VI due to distinct mode phase difference. Hence, the manipulation of the mode phase difference is crucial for the uniformity of the output beam. Furthermore, both uniformly focusing and maximized DOF gain are realized in case III by the magnified MIF approach, which is thus chosen for the proposed probe. Compared to the existing probe design, the proposed probe increases the WD from 130 μm to 200 μm in air.

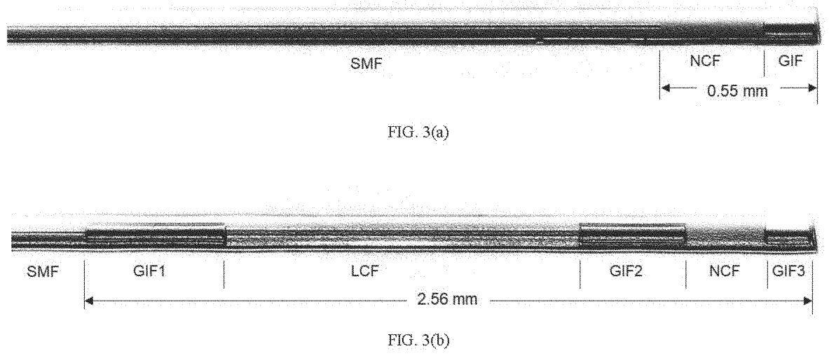

To fabricate the probe based on case III, each optical fiber is sequentially cut by a fiber cleaver and spliced to the end of the probe by a fiber fusion splicer. A conventional probe with the same lateral resolution was also fabricated for comparison, as shown in FIG. 3(a). In order to reduce the reflection of the probe end face, a short eight-degree angle-cleaved NCF was attached to the ends of the above two probes. FIG. 3(b) shows the microscope images of the two fabricated probes. The distal optics consisting by segmented fibers with the same diameter as that of a standard SMF make the probe robust in mechanical property and flexible for applications.

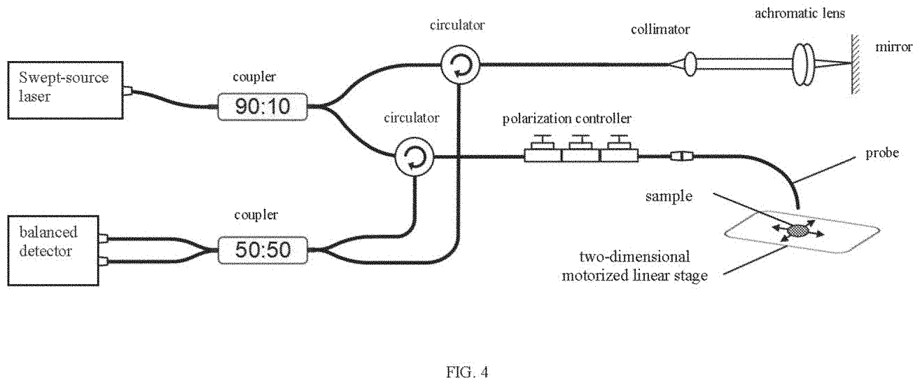

In order to compare and illustrate the advantages of the probe of the present application in OCT imaging, the above two probes were connected to the established swept source OCT system, as shown in FIG. 4. The central wavelength of the swept source is 1.3 μm and the bandwidth is 100 nm. In order to achieve two-dimensional or three-dimensional imaging, the probe is kept stationary, while the sample is placed on a two-dimensional motorized linear stage for lateral scanning. The collected interference spectrums are uniformly sampled in the wavenumber domain, and the dispersion compensation algorithm as well as the fast Fourier transform are applied to obtain OCT images.

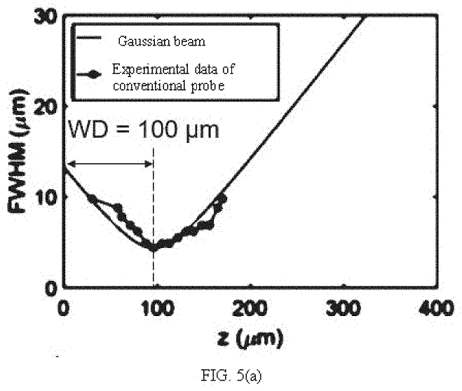

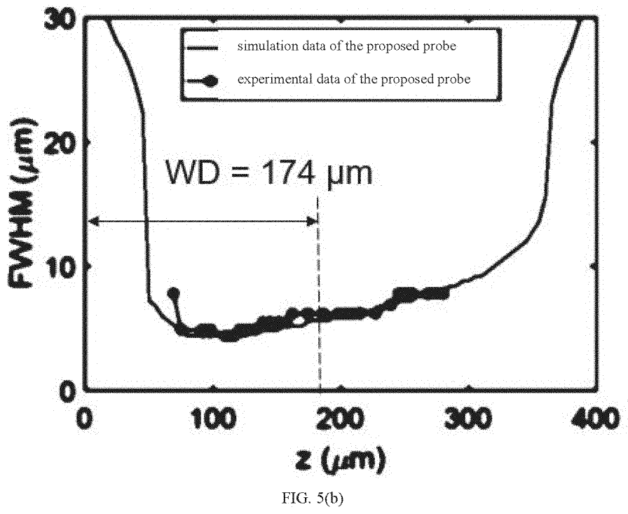

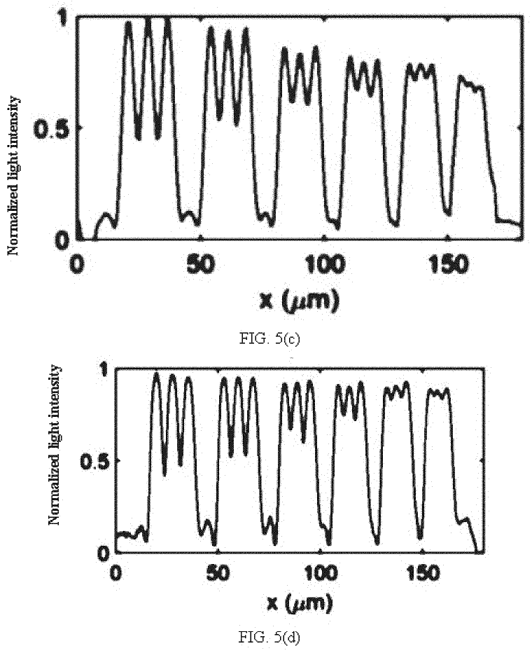

The diameters and DOFs of the output beams are calibrated by OCT imaging on a 1951 USAF resolution test target. Elements in group 6, 7 of the test target are chosen, corresponding to line pair period from 4.4 μm to 13.9 μm. The beam diameters versus depth are plotted in FIG. 5(a) for the conventional probe and FIG. 5(b) for the proposed probe, corresponding to a DOF of 103 μm and 211 μm, respectively. The target's reflectivity profiles at the probes' foci are given in FIGS. 5(c) and (d), indicating that the minimized lateral resolutions for two probes are similar and better than 4.4 μm. Thus, it can be concluded that, compared with the conventional probe, two times of DOF gain is achieved by the proposed probe without loss of lateral resolution. Also, it can be observed from FIG. 5(a)(b) that the measured WD is 100 μm for the conventional probe and 174 μm for the proposed probe. Both WDs are slightly shortened due to the mentioned appended NCFs at the end of the probes. Furthermore, no degradation in axial resolution is observed in the proposed probe within its DOF range, and the measured axial resolutions for both probes are 11.3 μm.

A probe for OCT that utilizes fiber mode interference to simultaneously achieve focal depth extension, working distance extension, and axial light intensity uniformity optimization is provided. The diameter and the length of the distal optical component of the probe is 125 μm and 2.6 mm, respectively. Compared with the conventional probe with the same lateral resolution (better than 4.4 μm), the probe of the present application has twice the depth of focus and 1.7 times the working distance. Due to the advantages of optimized imaging quality, easy manufacturing, reliable structure and flexible application scenarios, the probe of the present application has potential application in important fields.

1. A probe with optimized focal depth, working distance and axial light intensity uniformity, comprising a single-mode fiber, a first gradient index fiber, a large core fiber, a second gradient index fiber, a no-core fiber and a third gradient index fiber,
the single-mode fiber, the first gradient index fiber, the large core fiber, the second gradient index fiber, the no-core fiber, and the third gradient index fiber are spliced by fusion in sequence; the second gradient index fiber magnifies a MIF (Mode Interference Field) on an interface between the large core fiber and the second gradient index fiber to an entrance pupil of the third gradient index fiber, a length of the no-core fiber satisfies the requirement that the magnified MIF fully fills the aperture of the third gradient index fiber.

2. The probe with optimized focal depth, working distance and axial light intensity uniformity according to claim 1, wherein the MIF is generated at an end of the large core fiber, the MIF is adjusted by a length of the first gradient index fiber and a length of the large core fiber; the first gradient index fiber adjusts a mode energy of the MIF, and the large core fiber adjusts a mode phase difference of the MIF.

3. The probe with optimized focal depth, working distance and axial light intensity uniformity according to claim 1, wherein the length of the first gradient index fiber is zero.

4. The probe with optimized focal depth, working distance and axial light intensity uniformity according to claim 1, wherein an outer diameter of each fiber is the same as that of a standard single-mode optical fiber.

5. The probe with optimized focal depth, working distance and axial light intensity uniformity according to claim 1, wherein a length of the third gradient index fiber used for focusing realizes a lateral resolution required by an OCT system.
